(19) United States
(12) Patent Application Publication
(10) Pub. No.: US 2003/0203367 A1
(43) Pub. Date: Oct. 30, 2003

(54) Novel nomenclature or use of specific digital assignments to the bases in the deoxyribonucleic acid (DNA) and ribonucleic acid (RNA)

(76) Inventor: Hasmukh B. Patel, Edison, NJ (US)

Correspondence Address: HASMUKH B. PATEL, 44 HANA ROAD, EDISON, NJ, 08817, US

(21) Appl. No.: 10/134,952

(22) Filed: Apr. 29, 2002

(51) Int. Cl. C12Q 1/68; G06F 19/00; G01N 33/48; G01N 33/50

(52) U.S. Cl. 435/6; 702/20

(57) ABSTRACT

The current invention is centered on the use of numerical (digital) assignments using 0, 1, 2 and 3 (used both for T & U) to the four bases A, C, G, and T respectively, for DNA and A, C, G & U for RNA. It is claimed in the current invention (referred as Patel System) that the use of numerical assignment will allow a better scientific representation and or understanding of the genome information and also that it will reduce the data storage requirement as well as faster data processing of the genome information.

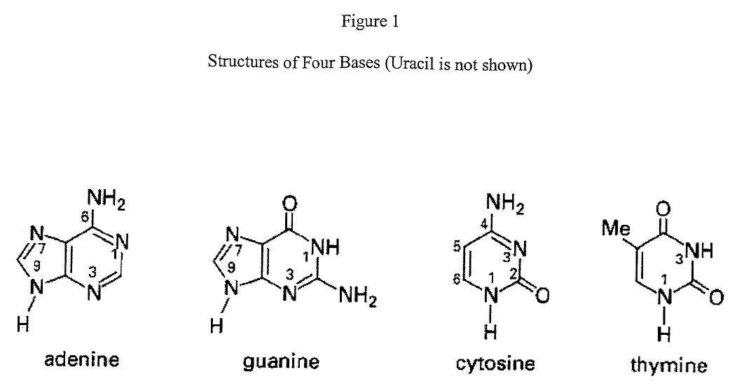

### BRIEF SUMMARY OF THE INVENTION

The current invention is centered on the use of numerical (digital) assignments 0, 1, 2 and 3 (used both for T & U) to the four bases A, C, G, and T respectively, for DNA and A, C, G & U for RNA. It is claimed in the current invention (of digital assignment is referred in this document as Patel System of Nomenclature) that the use of numerical assignment will allow a better scientific representation and or understanding of the genome information and also that it will reduce the data storage requirement as well as faster data processing of the genome information.

Following are the claimed benefits of the use of Patel System:

1. Better Scientific Presentation[Understanding of Genome Information:

Table 1 represents the basic assumption and a comparison of the nomenclature of codons representation of amino acid using the Old Nomenclature and the newly invented Patel System nomenclature. It is apparent that the digital assignment makes the codon more ordered as compared to the alphabetical system. Additionally, as shown in Table 2 the organization of the information on amino acid codes is having a definite order because of the application of Patel System.

2. Reduced Data Storage Requirement and data access time:

The Patel System offers a numerical presentation of each base and allows computer storage using only two memory bits as an unsigned integer as compared to four bits that are required for the storage an alphabetical letter in a string format. Thus the storage requirement is cut in half allowing more genome data to be stored in a smaller sized disk storage. Therefore, instead of using 3/2 gigabytes it will require only 3/4 gigabytes. Additionally, due to the reduced storage requirements the access time for retrieval of the data is also reduced in half.

3. Faster Data Processing:

As explained in the item (2) above, the data storage for Patel System requires only 2 bits and as a consequence of this, twice as much data can be stored in the random access memory (RAM). This allows the central processing unit (CPU) to access more information for a comparison between supplied base sequence with the stored base sequence. Additionally, since the comparison is done only between two bits of data versus four bits of the data the processing speed is doubled resulting in an over all increase of more than double.

TABLE 1

Basic assumptions and the application of old and new nomenclature
to the amino acids

| | |
|---|---|
| Axiom: The sum of the | Patel |
| Pairing bases is 3 | System |

| Base | Codes | Sum | | Base | Code |
|---|---|---|---|---|---|
| CG | 12 | 3 | A | 0 | |
| AT (or AU) | 03 | 3 | C | 1 | |
| | | | G | 2 | |
| | | | T or U | 3 | |

| | |
|---|---|
| Old System of Nomenclature | New System of Nomenclature |
| Codon by Conventional System | Codon by Patel System |

| Amino acid | Abbr. | Code | 1 | 2 | 3 | 4 | 5 | 6 | 1′ | 2′ | 3′ | 4′ | 5′ | 6′ | |
|---|---|---|---|---|---|---|---|---|---|---|---|---|---|---|---|
| Lysine | Lys | K | AAA | AAG | | | | | 000 | 002 | | | | | |
| Asparagine | Asn | N | AAC | AAT | | | | | 001 | 003 | | | | | |
| Threonine | Thr | T | ACA | ACC | ACG | ACT | | | 010 | 011 | 012 | 013 | | | |
| Arginine | Arg | R | AGA | AGG | CGA | CGC | CGG | CGT | 020 | 022 | 120 | 121 | 122 | 123 | |
| Serine | Ser | S | AGC | AGT | TCA | TCC | TCG | TCT | 021 | 023 | | 310 | 311 | 312 | 313 |
| Isoleucine | Ile | I | ATA | ATC | ATT | | | | 030 | 031 | 033 | | | | |
| Methionine | Met | M | ATG | | | | | | 032 | | | | | | |
| Glutamine | Gln | Q | CAA | CAG | | | | | 100 | 102 | | | | | |
| Histidine | His | H | CAC | CAT | | | | | 101 | 103 | | | | | |
| Proline | Pro | P | CCA | CCC | CCG | CCT | | | 110 | 111 | 112 | 113 | | | |
| Glutamic acid | Glu | E | GAA | GAG | | | | | 200 | 202 | | | | | |
| Aspartic acid | Asp | D | GAC | GAT | | | | | 201 | 203 | | | | | |
| Alanine | Ala | A | GCA | GCC | GCG | GCT | | | 210 | 211 | 212 | 213 | | | |
| Glycine | Gly | G | GGA | GGC | GGG | GGT | | | 220 | 221 | 222 | 223 | | | |
| Valine | Val | V | GTA | GTC | GTG | GTT | | | 230 | 231 | 232 | 233 | | | |
| End | End | * | TAA | TAG | | | | | 300 | 302 | | | | | |
| Tyrosine | Tyr | Y | TAC | TAT | | | | | 301 | 303 | | | | | |
| End | End | ** | TGA | | | | | | 320 | | | | | | |
| Cysteine | Cys | C | TGC | TGT | | | | | 321 | 323 | | | | | |
| Tryptophan | Trp | W | TGG | | | | | | 322 | | | | | | |
| Leucine | Leu | L | TTA | TTG | CTA | CTC | CTG | CTT | 330 | 332 | 130 | 131 | 132 | 133 | |
| Phenylalanine | Phe | F | TTC | TTT | | | | | 331 | 333 | | | | | |

TABLE 2
Base Position Table for the Amino Acids Indicated by Their Codes

| 1st\2nd | 0 | 1 | 2 | 3 | 3rd |
|---|---|---|---|---|---|
| 0 | K | T | R | I | 0 |
| | N | T | S | I | 1 |
| | K | T | R | M | 2 |
| | N | T | S | I | 3 |
| 1 | Q | P | R | L | 0 |
| | H | P | R | L | 1 |
| | Q | P | R | L | 2 |
| | H | P | R | L | 3 |
| 2 | E | A | G | V | 0 |
| | D | A | G | V | 1 |
| | E | A | G | V | 2 |
| | D | A | G | V | 3 |
| 3 | END | S | END | L | 0 |
| | Y | S | C | F | 1 |
| | END | S | W | L | 2 |
| | Y | S | C | F | 3 |

Where, 1^{st}, 2^{nd}, and 3^{rd} refers to the position of the base in the codon corresponding to the amino acid code in the given column and row.

1. A new invention ((Patel System) of specific digital assignment (see SPECIFICATION) of the bases in Nucleic Acid sequence is claimed.
0 for Adenine,
1 for Cytosine,
2 for Guanine, and
3 for Thymine or Uracil
or a numerical assignment to any base in any order.
Any unauthorized use of digital (numerical) assignment of the bases/nucleotides should not be allowed without prior authorization by the inventor.

2. It is also claimed that this invention will allow the following:
a. Will reduce requirements for the computational storage of sequence information.
b. Will improve the speed of computational processing due to reduced random access memory requirement as well as due to the numerical comparison instead of alphabetical comparison.
c. Will simplify the computation for the generation of complementary copies of the DNA or RNA or a fragment thereof and will increase the speed of data processing.
d. Will help better interpretation/understanding of the base sequence/codons (see Table 2) as there is a mathematical basis for the numerical assignment to the bases instead of arbitrary alphabetical assignments.
